(19) United States
(12) Patent Application Publication
(10) Pub. No.: US 2009/0137558 A1
(43) Pub. Date: May 28, 2009

(54) Pyridine based compounds useful as intermediates for pharmaceutical or agricultural end-products

(75) Inventors: Raymond Fisher, Derbyshire (GB); Andrew Lund, Derbyshire (GB)

Correspondence Address: MORGAN, LEWIS & BOCKIUS LLP (SF), One Market, Spear Street Tower, Suite 2800, San Francisco, CA, 94105, US

(73) Assignee: PEAKDALE MOLECULAR LIMITED, Derbyshire (GB)

(21) Appl. No.: 11/792,181

(22) PCT Filed: Dec. 1, 2005
(86) PCT No.: PCT/GB2005/004596

§ 371 Date: Apr. 4, 2008

Related U.S. Application Data
(60) Provisional application No. 60/633,370, filed on Dec. 3, 2004.

(30) Foreign Application Priority Data
Dec. 3, 2004 (GB) 0426576.5

(51) Int. Cl. A61K 31/55; C07D 213/80; A61K 31/44; C07D 401/04; A61K 31/444; C07D 215/16; A61K 31/47; A61K 31/435; A61K 31/4375; C07D 221/16; C07D 471/02; C07D 403/04; A61K 31/497

(52) U.S. Cl. 514/217.07; 546/318; 546/322; 514/356; 546/257; 514/334; 546/178; 514/311; 514/290; 514/300; 546/111; 546/123; 544/362; 540/597; 514/253.04

(57) ABSTRACT

The present invention relates to substituted pyridine compounds of Formula (I) and derivatives thereof, and to a process for preparing these substituted pyridines. The invention also relates to the use of the substituted pyridines as intermediates in the production of pharmaceutical, chemical and agro-chemical products.

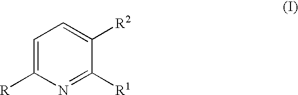

The present invention relates to substituted pyridines and derivatives thereof, and to a process for preparing these substituted pyridines. The invention also relates to the use of the substituted pyridines as intermediates in the production of pharmaceutical, chemical and agro-chemical products.

A number of routes are known for the synthesis of substituted pyridines. Okada et al (Okada et al., Heterocycles, 46, 129-(1997)) have shown that beta-trifluorovinylamine reacts with various methylene compounds to give substituted 6-(trifluoromethyl)nicotinic acids and closely related compounds. Bagley et al (Bagley et al., J Chem Soc, Perkin Trans 1, 1663 (2002)) have added to the scope of the Bohlmann-Rahtz reaction (Bohlmann and Rahtz Chem Ber, 90, 2265 (1957)) and described the synthesis of a small range of 2,6-disubstituted nicotinic esters and some of their derivatives. However, both of these syntheses are multi-step processes which start from commercially available materials and rely on harsh conditions for the final cyclisation namely a trifluoroacetic acid/benzene reflux (Okada et al., Heterocycles, 46, 129 (1997)) and a Lewis acid/toluene reflux (Bagley et al., J Chem Soc, Perkin Trans 1, 1663 (2002)).

The inventors have provided further substituted pyridines compounds. These compounds are prepared by a simplified process involving the mixing of commercially reagents in acetic acid followed by reflux. The compounds are useful as, or in the synthesis of, inter alia, pharmaceutical, nutraceutical or agricultural products.

According to a first aspect of the present invention there is provided a compound, or derivative thereof, of formula I

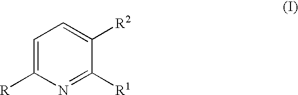

wherein R and R¹ are the same or different and R is a fluorinated C1-6 alkyl, COR³, CO₂R³, (CH₂)nCO₂R³ or (CH₂)nOR³ group optionally substituted by one or more of hydrogen, C1-6 alkyl or C1-6 haloalkyl;
R¹ is NR³R³ or hydrocarbyl optionally substituted by one or more of halogen (F, Cl, Br, I), CO₂R⁴, OR⁴, (CH₂)nOR⁴, (CH₂)nCO₂R³, NR⁴R⁴ or optionally substituted C1-6 alkyl;
R² is halogen, C1-6 alkyl (preferably methyl or ethyl), NO₂, CN, S(O)₂R³, COR³, CO₂R³, (CH₂)nOR³, (CH₂)nCO₂R³ or CONR³₂ optionally substituted by one or more of halogen (F, Cl, Br, I), OR⁴, CN, C1-6 alkyl, CO₂R⁴, (CH₂)nOR⁴, (CH₂)nCO₂R³ or NR⁴R⁴; wherein each saturated carbon in R² is further optionally and independently substituted by ═O, ═S, ═NR⁵, NNR⁵₂ or ═NOR⁵;
or R¹ and R² together form a partially saturated, unsaturated or fully saturated five or six membered ring containing zero to three heteroatoms which is further optionally fused to another partially saturated, unsaturated or fully saturated five or six membered ring to form a ring system containing zero to three heteroatoms, and each substitutable carbon atom in the optionally fused ring(s) or ring system(s) is optionally and independently substituted by one or more of halogen (Cl, I, F or Br), ═O, ═S, C1-12 alkyl (e.g C1-6 alkyl), C1-12 haloalkyl, cyclohydrocarbyl, heterocyclyl, OR³, SR³, NR³₂, NO₂, CN, NR³COR³, NRCONR³₂, NRCOR³, NR³CO₂R³, S(O)₂R³, SONR³₂, S(O)₂R³, SO₂NR³₂, NR³S(O)₂R³, COR³; CO₂R³, (CH₂)nOR³, (CH₂)nCO₂R³ or CONR³₂ optionally substituted by one or more of halogen (F, Cl, Br, I), optionally substituted C1-6 alkyl, CO₂R⁴, (CH₂)nOR⁴, (CH₂)nCO₂R⁴ or NR⁴R⁴;
R³ which may be the same or different and is hydrogen, halogen (Cl, F, I or Br), CN, OR⁵, CO₂R⁵, (CH₂)nNR⁵R⁵, NR⁵R⁵, (CH₂)nOH, C1-6 alkyl (e.g methyl or ethyl), heterocyclyl or aryl;
R⁴ which may be the same or different is hydrogen, halogen, CN, OR⁵, (CH₂)nNR⁵R⁵, NR⁵R⁵, optionally substituted C1-12 alkyl (e.g C1-6), heterocyclyl or aryl;
R⁵ which may be the same or different is hydrogen, halogen (Cl, F, I or Br), C1-6 alkyl or C1-6 haloalkyl;
wherein n is 0 to 6, preferably 1, 2 or 3;
or a pharmaceutically acceptable salt, and other pharmaceutically acceptable derivatives thereof, and including the proviso that the compound is not methyl 2-methyl-6-(trifluoromethyl)-nicotinate, 2-methyl-6-(trifluoromethyl)nicotinic acid, 1-[2-methyl-6-(trifluoromethyl)pyridine-3-yl]ethanone; diethyl 6-methylpyridine-2,5-dicarboxylate, [2-methyl-6-(trifluoromethyl)pyridine-3-yl](phenyl)methanone or N-[7-(4-methoxyphenyl)-2-(trifluoromethyl)-1,6-naphthyridin-5-yl]propane-1,3-diamine.

For the purposes of this invention hydrocarbyl includes, but is not limited to, alkyl, alkenyl, alkynyl, vinyl, heterocyclyl, cyclohydrocarbyl, for example cycloalkyl, cycloalkenyl and moieties containing a combination thereof.

As used herein “alkyl” relates to both straight chain and branched alkyl radicals, for example, of 1 to 12 carbon atoms, e.g. 1, 2, 3, 4, 5, 6, 7, 8 carbon atoms including but not limited to methyl, ethyl, n-propyl, isopropyl, n-butyl, sec-butyl, isobutyl, tert-butyl, n-pentyl, n-hexyl, n-heptyl, n-octyl. The term alkyl also encompasses cycloalkyl radicals including but not limited to cyclopropyl, cyclobutyl, cyclopentyl or cyclohexyl.

The alkyl group may be substituted with one or more halogen atoms. In one class of compounds the halogen is fluorine and the alkyl group is mono-, di- or trifluoromethyl.

“Alkoxy” relates to both straight chain and branched alkyl radicals, for example, of 1 to 12 carbon atoms, e.g. 1, 2, 3, 4, 5, 6, 7, 8 carbon atoms containing one or more oxygen atoms or hydroxyl.

The term “alkenyl” means a straight or branched alkenyl radical of, for example, 2 to 12 carbon atoms, such as 2, 3, 4, 5 or 6 carbon atoms, and containing one or more carbon-carbon double bonds and includes but is not limited to ethylene, n-propyl-1-ene, n-propyl-2-ene, isopropylene etc.

“Alkynyl” relates to a straight or branched alkynyl radical of, for example, 2 to 12 carbon atoms, such as 2, 3, 4, 5 or 6 carbon atoms, and containing one or more triple bonds.

“Cyclohydrocarbyl” relates to a saturated, partly unsaturated or unsaturated 3-10, for example, 5, 6, 7, 8, 9 or 10, membered hydrocarbon ring, including cycloalkyl or aryl.

“Aryl” means an aromatic, for example, 6-10 membered hydrocarbon containing one, e.g. 6C-10C, ring which is optionally fused to one or more saturated or unsaturated rings, including phenyl or phenyl substituted by an alkyl or alkoxy group in which alkyl and alkoxy are as described herein.

“Heteroaryl” means an aromatic, for example, 5-10 membered aromatic ring containing one or more heteroatoms selected from N, O or S, and containing one ring which is optionally fused to one or more saturated or unsaturated rings.

“Heterocyclyl” means, for example, a 3-10 membered, for example, 5, 6, 7, 8, 9 or 10, ring system containing one or more heteroatoms selected from N, O or S and includes heteroaryl. The heterocyclyl system may contain one ring or may be fused to one or more saturated or unsaturated rings; the heterocyclyl may be fully saturated, partially saturated or unsaturated.

“Ring” encompasses unsaturated or partially unsaturated rings but is usually a saturated ring, typically containing 5 to 13 ring-forming atoms, for example a 5- or 6-membered ring. The ring(s) may in turn be fused to one or more other rings, e.g the five or six membered ring may be fused to a further five or six membered ring, to form a ring system. The ring or ring system may be a cyclohydrocarbyl or heterocyclyl group.

Examples of cyclohydrocarbyl or heterocyclyl groups include but are not limited to cyclohexyl, cyclopentyl, phenyl, acridine, benzimidazole, benzofuran, benzothiophene, benzoxazole, benzothiazole, carbazole, cinnoline, cyclohexanone, cyclopentanone, dioxin, dioxane, dioxolane, dithiane, dithiazine, dithiazole, dithiolane, furan, imidazole, imidazoline, imidazolidine, indole, indoline, indolizine, indazole, isoindole, isoquinoline, isooxazole, isothiazole, morpholine, napthyridine, oxazole, oxadiazole, oxathiazole, oxathiazolidine, oxazine, oxadiazine, phenazine, phenothiazine, phenoxazine, phthalazine, piperazine, piperidine, pteridine, purine, putrescine, pyran, pyrazine, pyrazole, pyrazoline, pyrazolidine, pyridazine, pyridine, pyrimidine, pyrrolidine, pyrrole, pyrroline, quinoline, quinoxaline, quinazoline, quinnolizine, tetrahydrofuran, tetrazine, tetrazole, thiophene, thiadiazine, thiadiazole, thiatriazole, thiazine, thiazole, thiomorpholine, thianaphthalene, thiopyran, triazine, triazole, trithiane, tropine.

Halogen means F, Cl, Br, or I.

In a preferred aspect of the invention R is fluorinated methyl or ethyl (preferably CF₃or CH₂F) or CO₂R³ wherein R³ is hydrogen or C1-6 alkyl (preferably methyl or ethyl). Preferably still R is CF₃.

In a preferred aspect of the invention R¹ is alkyl (preferably methyl), (CH₂)nCO₂R³, NR³R³, vinyl or aryl (preferably phenyl or pyridine) optionally substituted by one or more of halogen (preferably F or Cl); wherein R³ is H or C1-6 alkyl (preferably methyl); and wherein n is 1. Preferably still R¹ is methyl, chloromethyl, NH₂, pyridine, phenyl, fluorophenyl or dimethylaminovinyl.

In a preferred aspect of the invention R² is COR³, CO₂R³, (CH₂)nOR³, (CH₂)nCO₂R³, S(O)₂R³ or C1-6 alkyl (preferably methyl or ethyl) optionally substituted by CN, NH₂ or OH; wherein each saturated carbon in R² is further optionally and independently substituted by ═O; wherein R³ is hydrogen, C1-6 alkyl (preferably methyl, ethyl or butyl), NH₂, CN, OH or aryl (preferably phenyl); and wherein n is 0, 1 or 2.

In a preferred aspect of the invention R¹ and R² together are a group of formula II

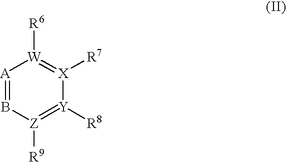

wherein A and B are C and form a bicyclic fused ring system with the ring of formula I; W, X, Y or Z are independently selected from N, O, C or S; R⁶, R⁷, R⁸ or R⁹ are independently selected from hydrogen, halogen (Cl, I, F or Br), ═O, ═S, B(OR¹²)₂, C1-12 alkyl (e.g C1-6 alkyl), C1-12 haloalkyl, cyclohydrocarbyl, heterocyclyl (preferably piperidine or piperazine), OR¹², SR¹², NR¹²₂, NO₂, CN, NR¹²COR¹², NRCONR¹²₂, NRCOR¹², NR¹²CO₂R¹², S(O)₂R¹², SONR¹²₂, S(O)R¹², SO₂NR¹²₂, NR¹²S(O)₂R¹², COR¹², CO₂R¹², (CH₂)nOR¹², (CH₂)nCO₂R¹² or CONR¹²₂ optionally substituted by one or more of halogen (F, Cl, Br, I), C1-6 alkyl, CO₂R¹³, (CH₂)nOR¹³, (CH₂)nCO₂R¹³, NR¹³R¹³ or heterocyclyl (preferably piperidine or piperazine) optionally substituted by NH₂; wherein each saturated carbon in R⁶, R⁷, R⁸ or R⁹ is further optionally and independently substituted by ═O, ═S, ═NR¹⁴, NNR¹⁴₂ or ═NOR¹⁴; or wherein any two of R⁶, R⁷, R⁸ or R⁹ form a partially saturated, unsaturated or fully saturated optionally substituted five or six membered ring containing zero to three heteroatoms (e.g N);
wherein R¹² which may be the same or different is hydrogen, halogen (Cl, F, I or Br), CN, OR¹⁵, CO₂R¹⁵, NR¹⁵R¹⁵, C1-6 alkyl (e.g methyl or ethyl) or heterocyclyl (preferably piperidine or piperazine);
wherein R¹³ which may be the same or different is hydrogen, halogen, CN, OR¹⁶, NR¹⁶R¹⁶, optionally substituted C1-12 alkyl (e.g C1-6);
R¹⁴, R¹⁵ and R¹⁶ are hydrogen or OH; and wherein n is 1 to 6, preferably 1 or 2.

R¹ and R² may together be optionally substituted pyridine, pyridazine, pyrimidine, pyrazine, pyran, quinoline, isoquinoline, quinazoline, pteridine, quinolizidine, indole, isoindole, indazole, purine or indolizidine. Preferably R¹ and R² together are substituted pyridine, pyrimidine, pyridazine or pyrazine. Preferably still R¹ and R² together are substituted pyridine or pyrimidine.

In a further preferred aspect of the invention one, two or three of W, X, Y and Z is other than C. Preferably still one or two of W, X, Y or Z is O or N, preferably N.

In a yet further preferred aspect R⁶, R⁷, R⁸ or R⁹ are independently selected from ═O, CN, halogen (Cl, Br or I), COR¹², CO₂R¹², NR¹²R¹², B(OR¹²)₂, (CH₂)nCO₂R¹², C1-6 alkyl (preferably methyl or ethyl), heterocyclyl (piperidine or piperazine) optionally substituted by one or more of NR¹³R¹³ or heterocyclyl (preferably piperidine);

wherein each saturated carbon in R⁶, R⁷, R⁸ or R⁹ is further optionally and independently substituted by ═O, ═S, ═NR¹⁴ or ═NOR¹⁴; wherein R¹² is hydrogen, halogen (preferably Br), NR¹⁵R¹⁵, C1-6 alkyl (preferably methyl), or heterocyclyl (preferably piperidine); wherein R¹³, R¹⁴ and R¹⁵ are hydrogen and wherein n is 1 or 2.

In a preferred aspect of the invention R¹ and R² are together a group of formula III

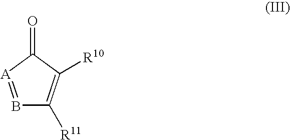

wherein A and B are C and form a bicyclic fused ring system with the ring of formula I; R¹⁰ and R¹¹ together optionally form a partially saturated, unsaturated or fully saturated optionally substituted six membered ring containing zero to three heteroatoms. Preferably still R¹⁰ and R¹¹ together are phenyl.

A compound according to the invention may be selected from the group consisting of

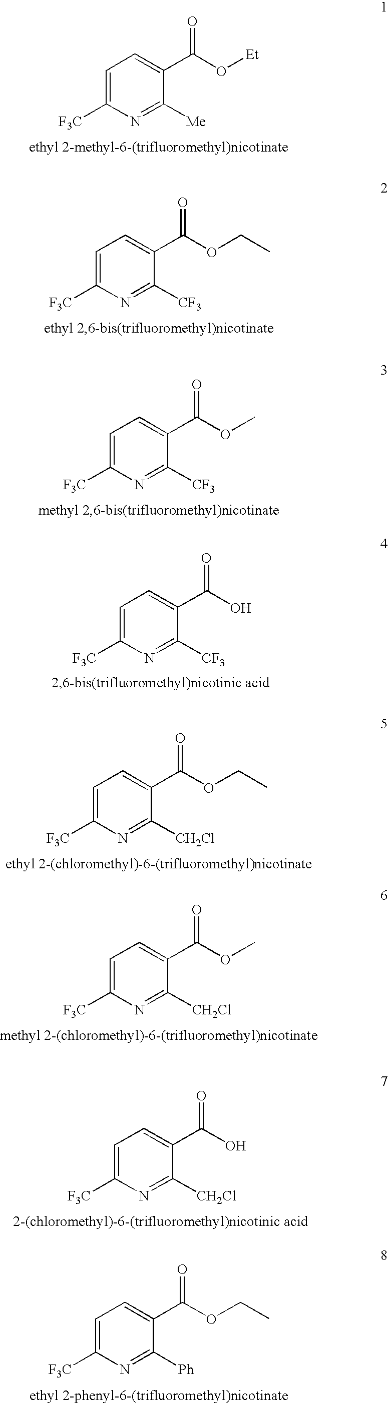

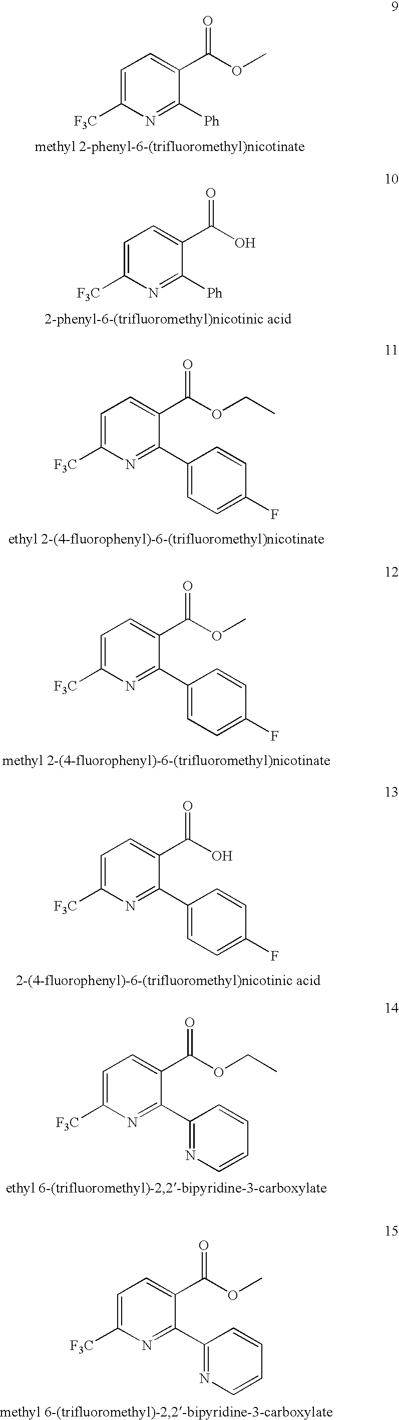

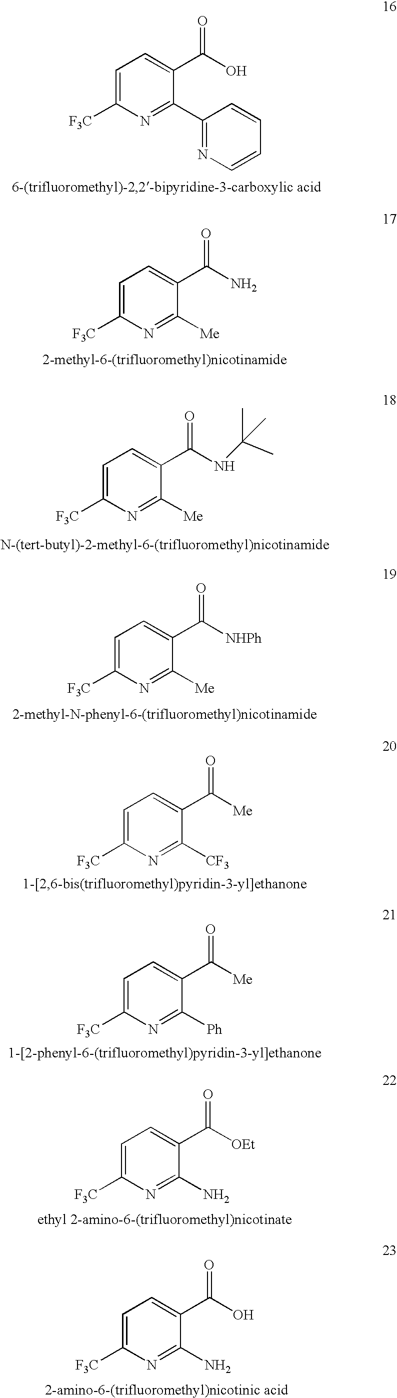

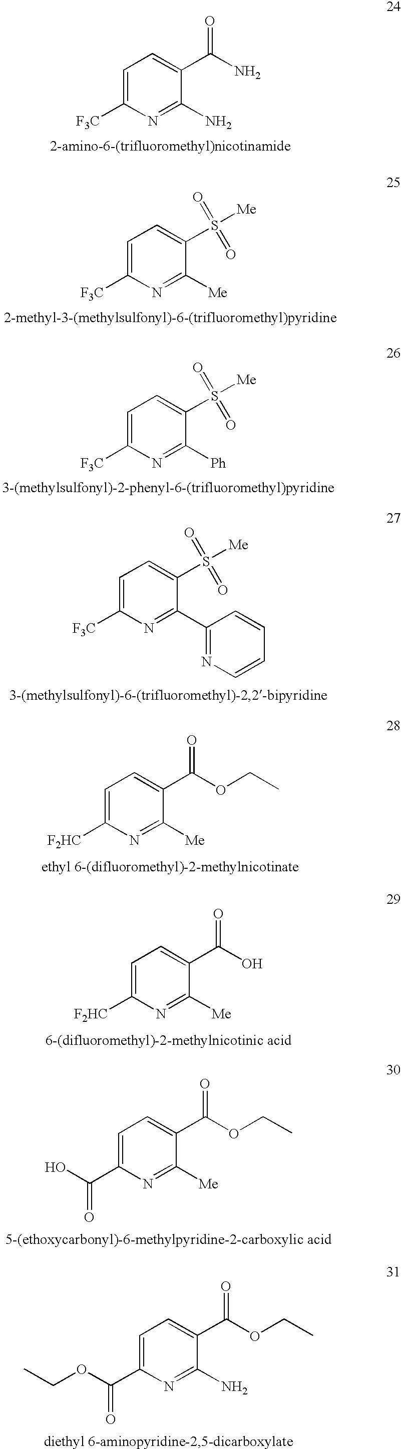

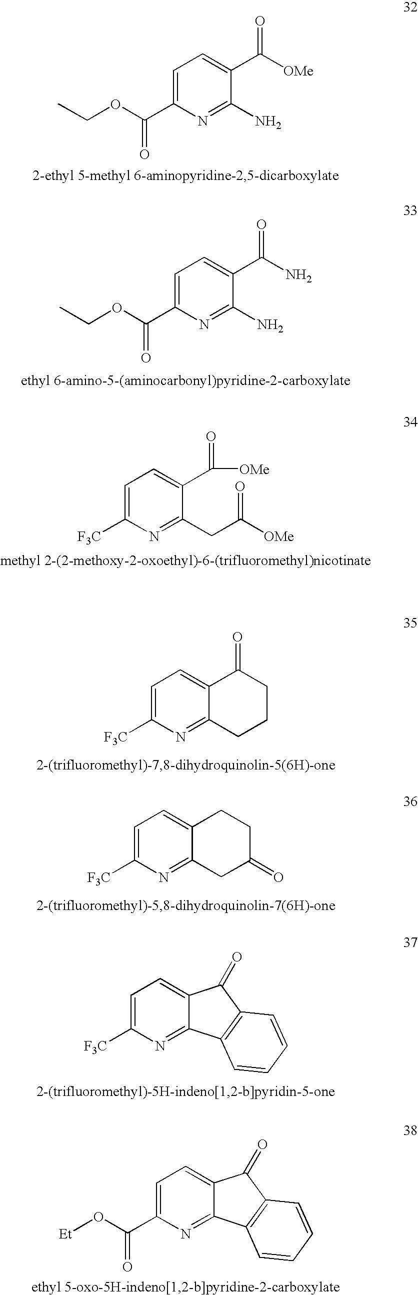

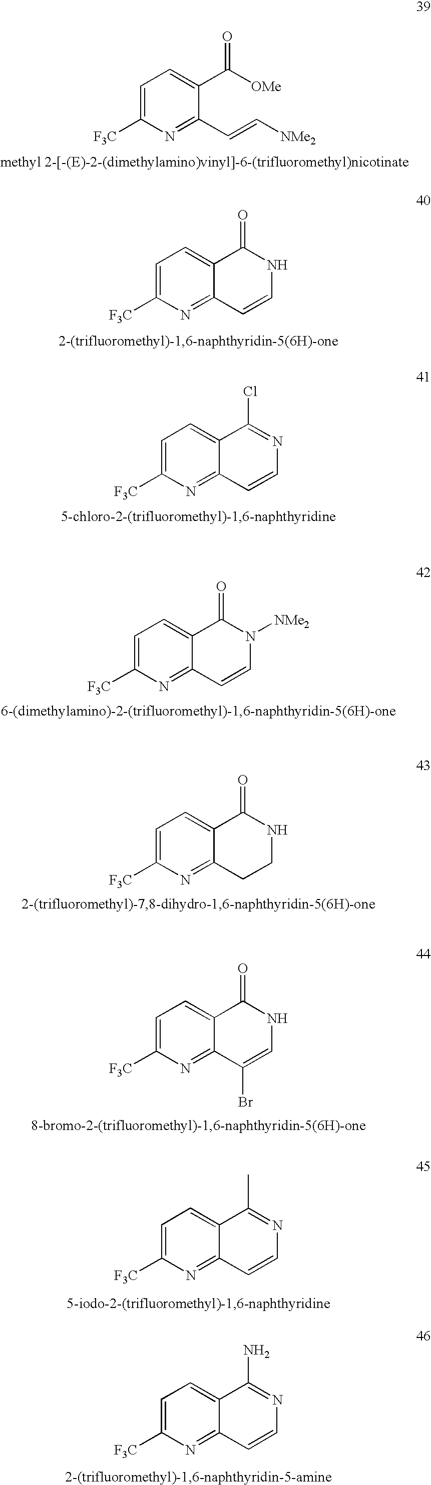

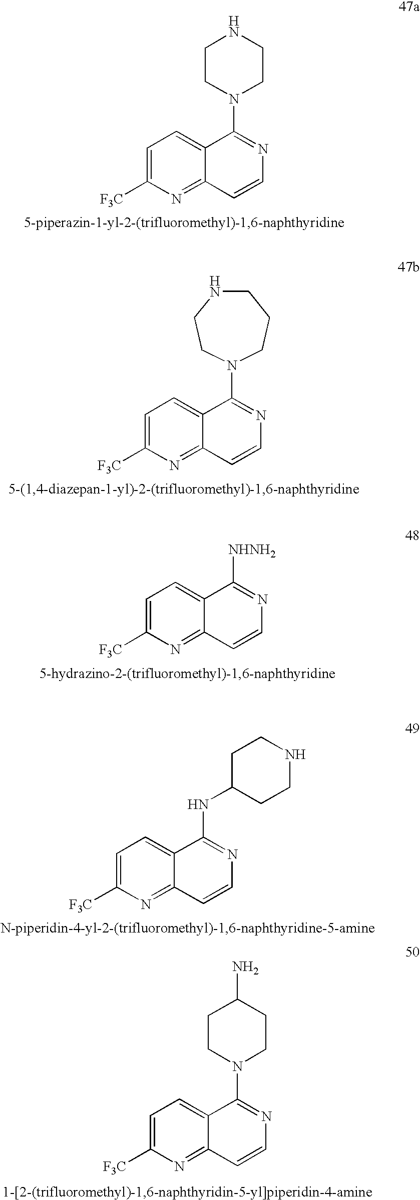

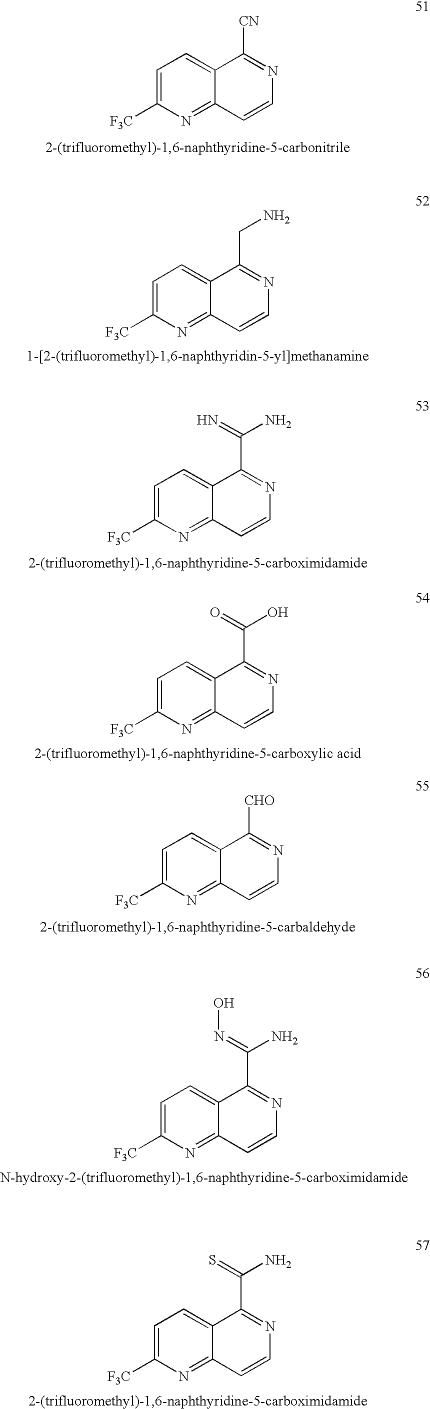

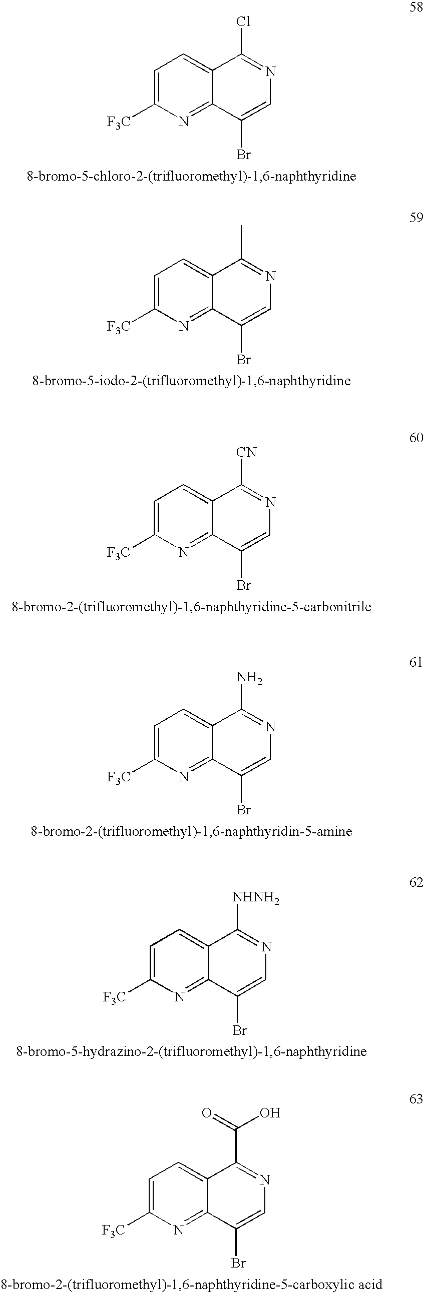

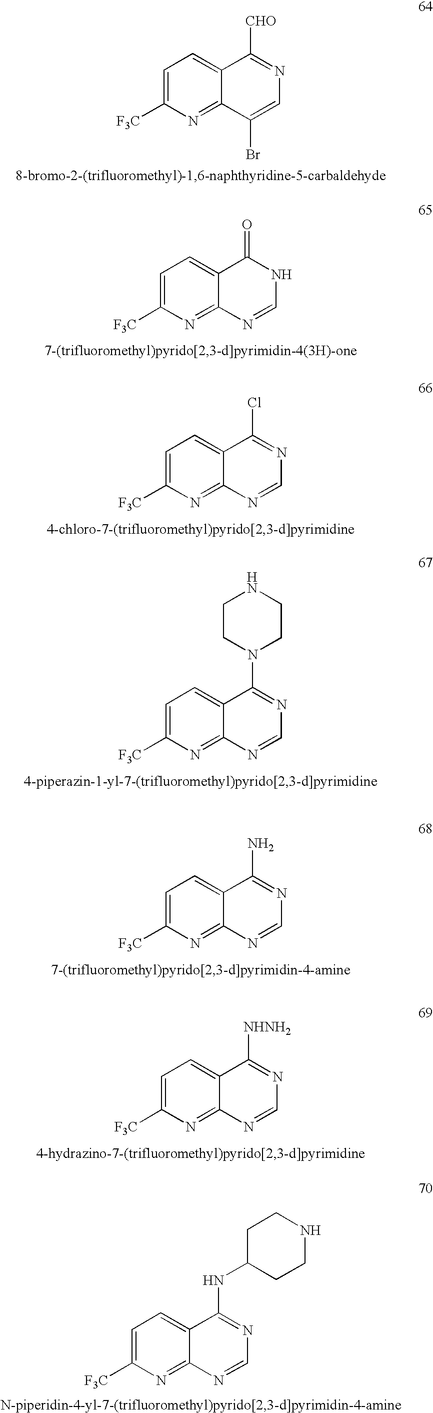

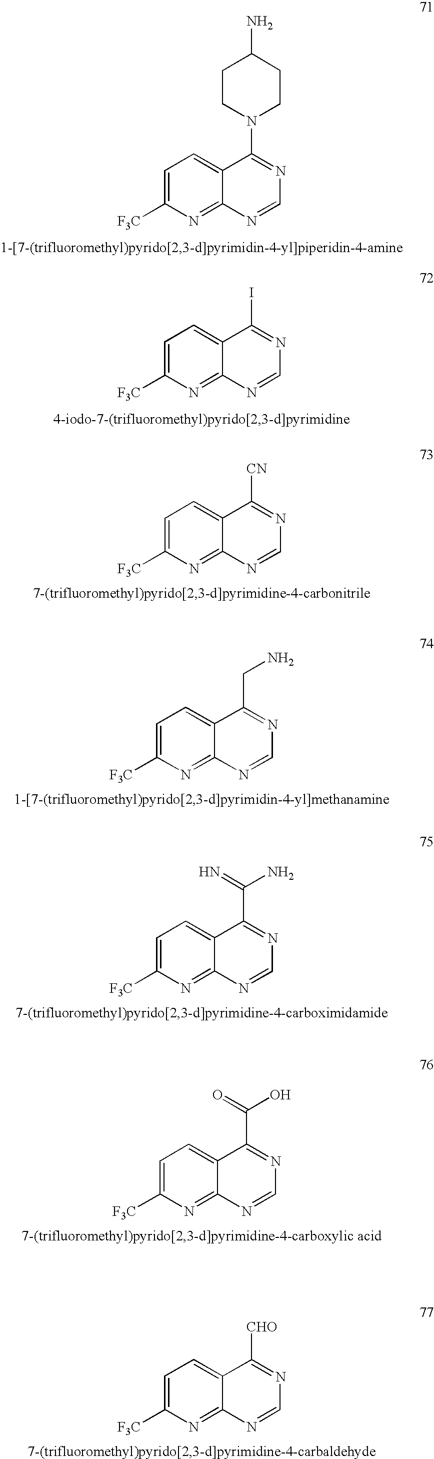

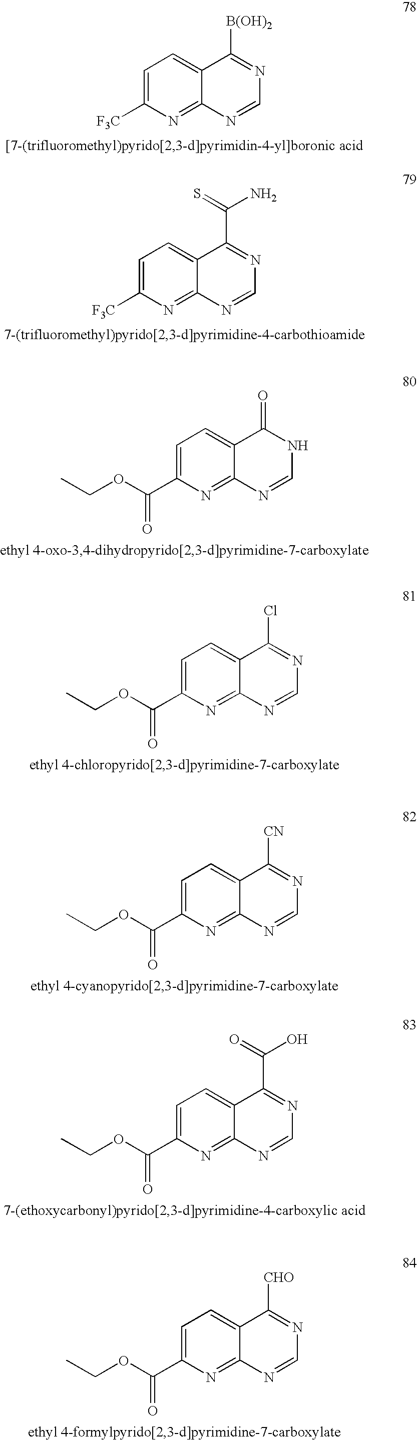

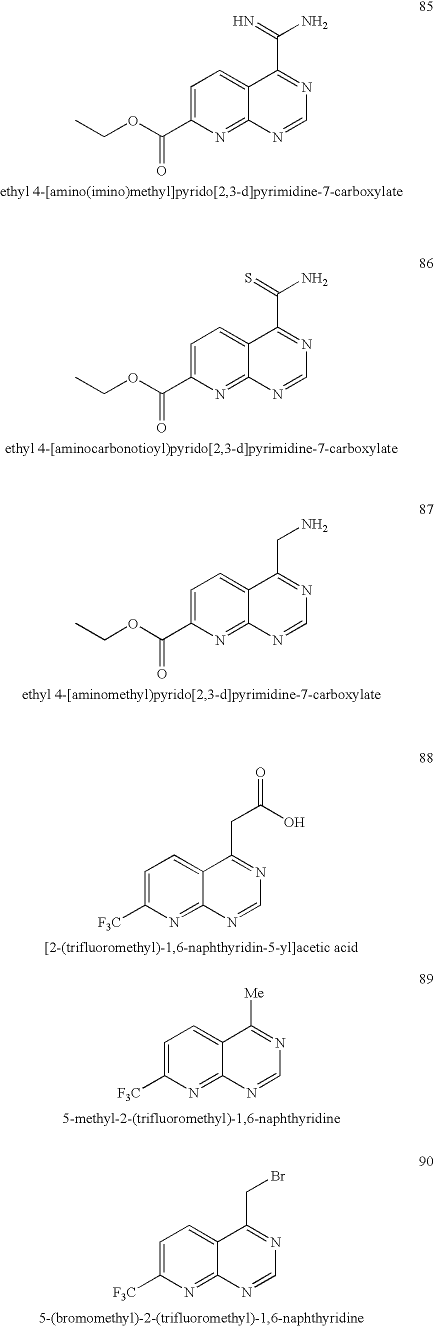

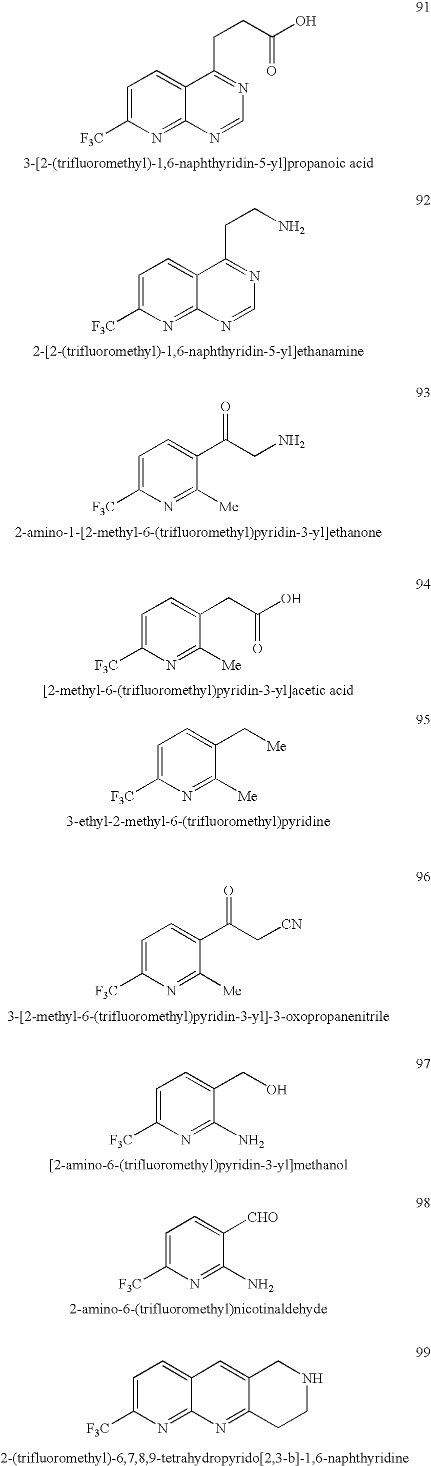

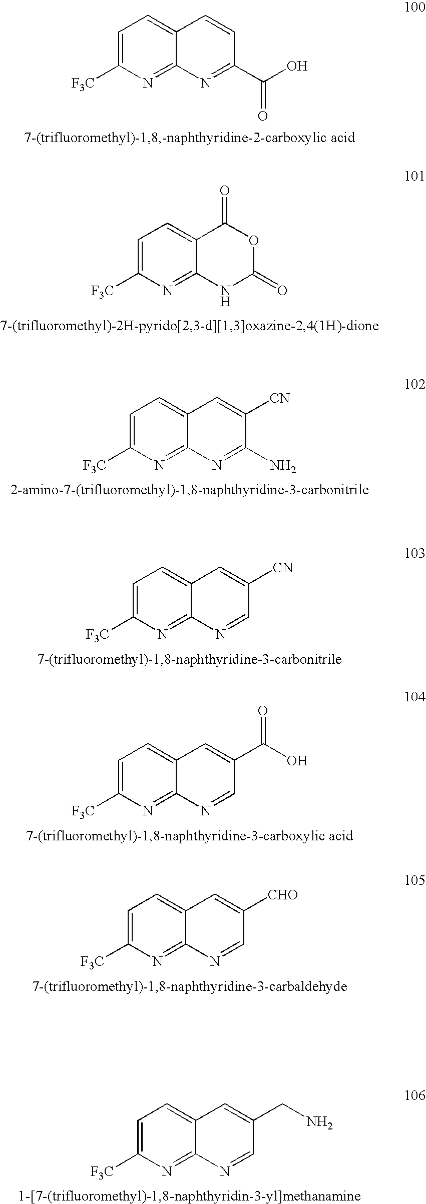

The compounds of the first aspect may be provided as a salt, preferably as a pharmaceutically acceptable salt of compounds of formula I. As used herein “pharmaceutically acceptable salts” is intended to mean salts which are compatible with pharmaceutical administration. Examples of pharmaceutically acceptable salts of these compounds include those derived from organic acids such as acetic acid, malic acid, tartaric acid, citric acid, lactic acid, oxalic acid, phosphoric, succinic acid, fumaric acid, maleic acid, benzoic acid, salicylic acid, phenylacetic acid, mandelic acid, methanesulphonic acid, benzenesulphonic acid and p-toluenesulphonic acid, mineral acids such as hydrochloric, hydrobromic, and sulphuric acid and the like, giving methanesulphonate, benzenesulphonate, p-toluenesulphonate, hydrochloride and sulphate, and the like, respectively or those derived from bases such as organic and inorganic bases. Examples of suitable inorganic bases for the formulation of salts of compounds for this invention include the hydroxides, carbonates, and bicarbonates of ammonia, lithium, sodium, calcium, potassium, aluminium, iron, magnesium, zinc and the like. Salts can also be formed with suitable organic bases. Such bases suitable for the formation of pharmaceutically acceptable base addition salts with compounds of the present invention include organic bases which are non-toxic and strong enough to form salts. Such organic bases are already well-known in the art and may include amino acids such as arginine and lysine, mono-, di-, or trihydroxyalkylamines such as mono-, di-, and triethanolamine, choline, mono-, di-, and trialkylamines, such as methylamine, dimethylamine, and trimethylamine, guanidine; piperidine, N-methylglucosamine; N-methylpiperazine; morpholine; ethylenediamine; N-benzylphenethylamine; tris(hydroxymethyl)aminomethane; and the like.

Salts may be prepared in a conventional manner using methods well known in the art. Acid addition salts of said basic compounds may be prepared by dissolving the free base compounds according to the first or second aspects of the invention in aqueous or aqueous alcohol solution or other suitable solvents containing the required acid. Where a compound of the invention contains an acidic function, a base salt of said compound may be prepared by reacting said compound with a suitable base. The acid or base salt may separate directly or can be obtained by concentrating the solution e.g. by evaporation. The compounds of this invention may also exist in solvated or hydrated forms.

The invention also extends to a prodrug of the aforementioned compounds such as an ester or amide thereof. A prodrug is any compound that may be converted under physiological conditions or by solvolysis to any of the compounds of the invention or to a pharmaceutically acceptable salt of the compounds of the invention. A prodrug may be inactive when administered to a subject but is converted in vivo to an active compound of the invention.

The present invention also provides derivatives including esters, amides, carbamates, carbonates, ureides, ureas, thioureas, hydantoins, thiohydantoins, diketopiperazines, solvates, hydrates, affinity reagents, peptides or prodrugs thereof.

A hydrolysable ester of a compound of the formula (I) containing a hydroxy group includes inorganic esters such as phosphate esters and acyloxyalkyl ethers and related compounds which as a result of in vivo hydrolysis of the ester break down to give the parent hydroxy group. Examples of acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters). Dialkylcarbamoyl and N-(N,N-dialkylaminoethyl)-N-alkylcarbamoyl (to give carbamates), N,N-dialkylaminoacetyl and carboxyacetyl. Examples of substituents on benzoyl include morpholino and piperazino.

A suitable example of a hydrolysable amide of a compound of the formula (I) containing a carboxy group is, for example, N—C₁₋₆alkyl amide or N,N-di-C₁₋₆alkyl amide such as N-methyl, N-ethyl, N-propyl, N,N-dimethyl, N-ethyl-N-methyl or N,N-diethyl amide.

According to a second aspect of the invention, there is provided a process for the manufacture of a compound of formula I which comprises reacting a compound of formula IV with a compound of formula V or VI optionally in the presence of an ammonia source

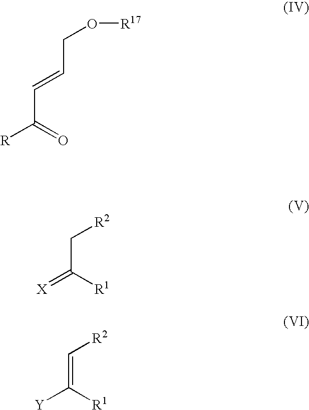

wherein, R, R¹ and R² are as hereinbefore defined;
R¹⁷ is hydrocarbyl preferably C1-12 alkyl (e.g. C1-6 alkyl);
X is O, NR¹⁸ or NR¹⁸₂ in protonated form and Y is NR¹⁸₂, wherein R¹⁸ is hydrogen;
wherein when X is O the reaction must be carried out in the presence of a source of ammonia.

Preferably, the ammonia source is an ammonium ion.

The process according to the invention may be carried out in the presence of acid or base catalysis, in the presence of a solvent and/or in the presence of microwaves.

Preferably when X is NR¹⁸₂ in protonated form R¹ is NR¹⁹R¹⁹ or OR¹⁹ wherein R¹⁹ is hydrogen or C1-12 alkyl (e.g C1-6 alkyl).

A further aspect of the invention provides an agent, for example, a pharmaceutical, nutraceutical, chemical or agrochemical agent comprising one or more compounds according to the invention.

The compounds according to the invention may be monomers for the preparation of polymers. Certain polymerisable compounds having polymerisable groups could be co-polymerised. Thus the agent may be a polymer or co-polymer.

The agent may be a dye. Alternatively, the agent may, for example, be a small molecule. Examples of small molecules include, but are not limited to, peptides, peptidomimetics (e.g., peptoids), amino acids and amino acid analogs.

The synthesis of peptides is well known in the art. Solid phase peptide synthesis generally proceeds by initial attachment of a first (alpha)-amino protected amino acid to a solid support (typically a resin) at its carboxylic end via a linker. Resins with certain protected amino acids already attached are available from commercial sources or can be synthesised by known methods. The (alpha) protecting group is removed from the resin linked amino acid and a second (alpha) amino acid protected amino acid is coupled to the first amino acid using a coupling agent. Cycles of deprotection and coupling of protected amino acids continue until the desired peptide sequence is prepared. The reaction conditions (reagent, solvent, concentration, temperature, time etc) of deprotection of the alpha amino protecting group selected for synthesis preferably do not cleave a substantial amount of the growing peptide from the resin selected for synthesis. Potentially reactive groups on the side chains of protected amino acid synthetic peptide building blocks may also be protected, typically with protecting groups that are not that are not substantially removed by the reaction conditions selected for removal of the (alpha) amino protecting group. A variety of protecting groups, reaction conditions for deprotection, coupling agents, reaction conditions for coupling linkers, resins and conditions for cleavage of the peptide from the resin are known in the art. Details of solid phase peptide synthesis are given, for example, in Greene and Wut, protecting groups in Organic synthesis, Wiley Science (1984) and later editions; Atherton and Sheppard (1989) in solid-phase peptide synthesis, A Practical Approach, IRL Press at Oxford University Press; Barany et al., (1987) Int. J. Peptide Protein Res 30: 705-739.

The compounds, salts and agents of the invention can be incorporated into pharmaceutical, nutraceutical or agricultural/agrochemical compositions.

Pharmaceutical or nutraceutical compositions typically include the compound, salt or agent along with a pharmaceutically or nutraceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical or nutraceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride, in the composition. Prolonged absorption of the injectable compositions can be brought about by including in the composition an agent which delays absorption, for example, aluminium monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troche or capsules, e.g., gelatin capsules. Oral compositions can also be prepared using a fluid carrier for use as a mouthwash.

Pharmaceutically compatible binding agents, and/or adjuvant materials can be included as part of the composition. The tablets, pills, troches, capsules and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

In one embodiment, the compounds may be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of compound calculated to produce the desired therapeutic effect in association with the required pharmaceutical carrier.

Exemplary doses include milligram or microgram amounts of the compound per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a compound depend upon the potency of the compound with respect to the expression or activity to be modulated. When one or more of these compounds is to be administered to an animal (e.g., a human), a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

In a further aspect of the invention, there is provided a compound according to the invention for use as a medicament.

In a further aspect of the invention, there is provided a compound according to the invention for use as in agriculture.

The invention will now be described by way of reference to the following non-limiting examples.

### EXAMPLE

### General Procedure

The structure of all compounds was confirmed by ¹H NMR (300 MHz) spectroscopy run on solutions in either deuterated chloroform or dimethylsulfoxide.

### Preparation of Ethyl 2-Methyl-6-(trifluoromethyl)nicotinate 1

### Method A

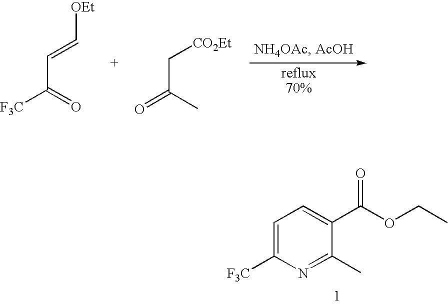

A mixture of (3E)-4-ethoxy-1,1,1-trifluorobut-3-en-3-one (100 g, 0.595 mol, 1.1 eq.) (note: (3E)-4-Ethoxy-1,1,1-trifluorobut-3-en-2-one was made according to the procedure described in R. J. Andrew, J. M. Mellor, G. Reid Tetrahedron 2000, 56, 7255), ethyl 3-oxobutanoate (70.4 g, 0.541 mol, 1.0 eq.), ammonium acetate (83.3 g, 1.08 mol, 2.0 eq.), and acetic acid (130 g, 2.17 mol, 4.0 eq.) was heated at reflux for 90 min and was then allowed to cool to room temperature. Water (300 mL) was added and the reaction mixture was extracted with dichloromethane (100 mL). The organic layer was separated, dried over magnesium sulphate, and the solvent was removed under reduced pressure to afford ethyl 2-methyl-6-(trifluoromethyl)nicotinate 1 (88.0 g, 70%) as a pale yellow oil, b.p. 56-62° C./0.25 mbar.

### Method B

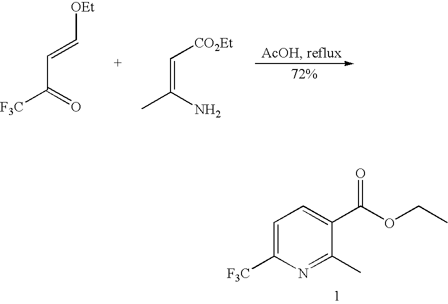

Ethyl 3-aminocrotonate (2.00 g, 15.5 mmol, 1.0 eq.) and (3E)-4-ethoxy-1,1,1-trifluorobut-3-en-2-one (2.60 g, 15.5 mmol, 1.0 eq.) were dissolved in glacial acetic acid (5 mL) and the resultant reaction mixture was heated at reflux for 1 h. The reaction was then allowed to cool to room temperature before being poured into water (100 mL) and extracted with hexane (50 mL). The organic layer was washed with water (2×20 mL), saturated aqeuous sodium bicarbonate (2×20 mL), and saturated aqueous sodium chloride (20 mL), and the solvent was removed under reduced pressure to afford ethyl 2-methyl-6-(trifluoromethyl)nicotinate 1 (2.59 g, 72%) as an oil.

### Preparation of Ethyl 2,6-Bis(trifluoromethyl)nicotinate 2

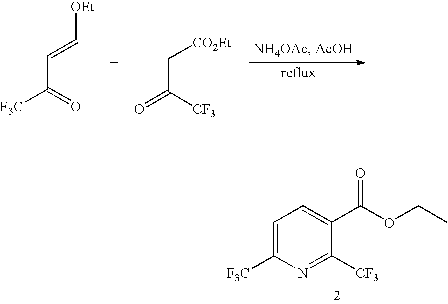

A mixture of (3E)-4-ethoxy-1,1,1-trifluorobut-3-en-3-one (10 g, 60 mmol, 1.0 eq.), ethyl 4,4,4-trifluoro-3-oxobutanoate (11.04 g, 60 mmol, 1.0 eq.), ammonium acetate (9.26 g, 120 mmol, 2.0 eq.), and acetic acid (14.4 g, 240 mmol, 4.0 eq.) was heated at reflux for 1 h and was then allowed to cool to room temperature. Water was added, the organic layer was separated, dried over magnesium sulphate, and the solvent was removed under reduced pressure to afford ethyl 2,6-bis(trifluoromethyl)nicotinate 2.

### Preparation of Ethyl 2-(Chloromethyl)-6-(trifluoromethyl)nicotinate 5

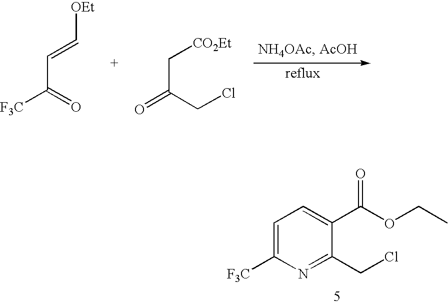

A mixture of (3E)-4-ethoxy-1,1,1-trifluorobut-3-en-3-one (10 g, 60 mmol, 1.0 eq.), ethyl 4-chloro-3-oxobutanoate (9.80 g, 60 mmol, 1.0 eq.), ammonium acetate (9.24 g, 120 mmol, 2.0 eq.), and acetic acid (14.4 g, 240 mmol, 4.0 eq.) was heated at reflux for 6 h and was then allowed to cool to room temperature. Water (100 mL) was added and the reaction mixture was extracted with dichloromethane (2×50 mL). The organic layer was separated, dried over magnesium sulphate, and the solvent was removed under reduced pressure. The crude product was purified by flash chromatography (silica, hexane) to afford ethyl 2-(chloromethyl)-6-(trifluoromethyl)nicotinate 5; R_{f} 0.80 (1:4 ethyl acetate/hexane).

### Preparation of Ethyl 2-Phenyl-6-(trifluoromethyl)nicotinate 8

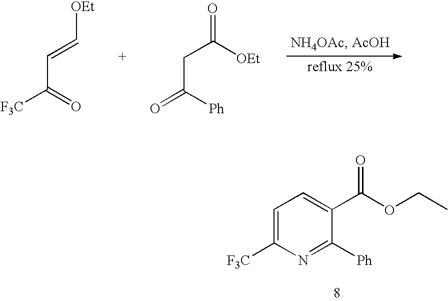

A mixture of (3E)-4-ethoxy-1,1,1-trifluorobut-3-en-3-one (50 g, 0.30 mol, 1.0 eq.), ethyl 3-oxo-3-phenylpropanoate (57.1 g, 0.30 mol, 1.0 eq.), and ammonium acetate (115 g, 1.5 mol, 5.0 eq.) in acetic acid (1.0 L) was heated at reflux overnight and then allowed to stand at room temperature for 1 week. Water (500 mL) was added and the reaction mixture was extracted with dichloromethane (2×200). The combined organic fractions were washed with water (3×100 mL), saturated aqueous sodium hydrogen carbonate, saturated aqueous sodium chloride, dried over magnesium sulphate, and the solvent was removed under reduced pressure. The residue was purified by distillation (Kugelrohr, 130° C./1 mbar) to afford ethyl 2-phenyl-6-(trifluoromethyl)nicotinate 8 (22.0 g, 25%) as an oil.

### Preparation of 2-Methyl-N-phenyl-6-(trifluoromethyl)nicotinamide 19

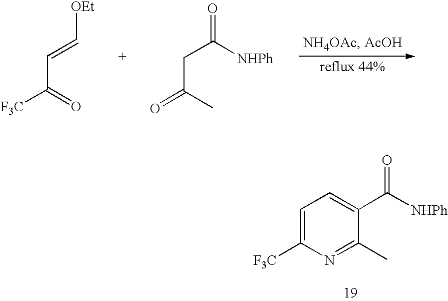

A mixture of (3E)-4-ethoxy-1,1,1-trifluorobut-3-en-3-one (5.0 g, 0.03 mol, 1.0 eq.), 3-oxo-N-phenylbutanamide (5.31 g, 0.03 mol, 1.0 eq.), ammonium acetate (4.02 g, 0.06 mol, 2.0 eq.), and acetic acid (18.0 g, 0.30 mol, 10.0 eq.) was heated at reflux overnight and then allowed to cool to room temperature. Water (50 mL) was added and the reaction mixture was extracted with hexane. The organic fraction was washed with saturated aqueous sodium chloride, dried over magnesium sulphate, and the solvent was removed under reduced pressure. The residue was purified by flash chromatography (silica, hexane then 10% ethyl acetate in hexane) and then recrystallized from heptane/ethyl acetate to afford 2-methyl-N-phenyl-6-(trifluoromethyl) nicotinamide 19 (3.7 g, 44%) as a crystalline solid, m.p. 161.1-161.4° C.

### Preparation of 1-[2,6-Bis(trifluoromethyl)pyridin-3-yl]ethanone 20

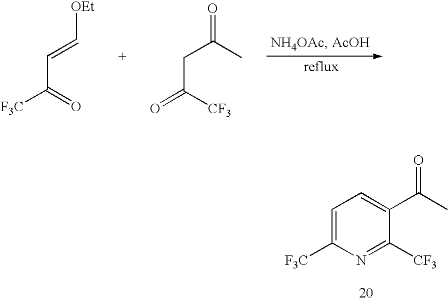

A mixture of (3E)-4-ethoxy-1,1,1-trifluorobut-3-en-3-one (5.0 g, 30 mmol, 1.0 eq.), 1,1,1-trifluoropentane-2,4-dione (4.62 g, 30 mmol, 1.0 eq.), ammonium acetate (4.62 g, 60 mmol, 2.0 eq.), and acetic acid (18 g, 0.3 mol, 10 eq.) was heated at reflux for 2 h and was then allowed to cool to room temperature. Water was added and the reaction mixture was extracted with hexane. The organic fraction was dried over magnesium sulphate and the solvent was removed under reduced pressure to give 1-[2,6-bis(trifluoromethyl)pyridin-3-yl]ethanone 20.

### Preparation of Methyl 2-(2-Methoxy-2-oxoethyl)-6-(trifluoromethyl)nicotinate 34

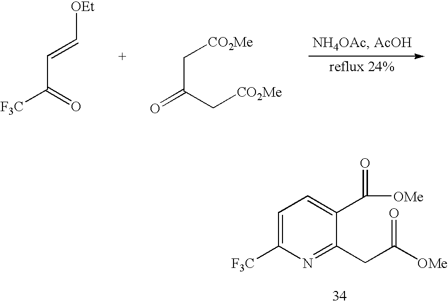

A mixture of (3E)-4-ethoxy-1,1,1-trifluorobut-3-en-3-one (2.41 g, 14.4 mmol, 1.0 eq.), dimethyl 3-oxopentanedioate (2.50 g, 14.4 mmol, 1.0 eq.), and ammonium acetate (5.54 g, 72.0 mmol, 5.0 eq.) in acetic acid (20 mL) was heated at reflux overnight and then allowed to cool to room temperature. Water (50 mL) was added and the reaction mixture was extracted with hexane (20 mL) then dichloromethane (20 mL). The combined organic fractions were dried over magnesium sulphate and the solvent was removed under reduced pressure to give methyl 2-(2-methoxy-2-oxoethyl)-6-(trifluoromethyl)nicotinate 34 (0.95 g, 24%).

### Preparation of 2-(Trifluoromethyl)-7,8-dihydroquinolin-5(6H)-one 35

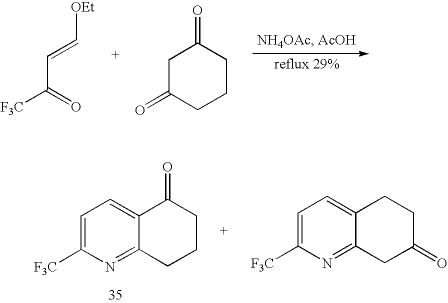

A mixture of (3E)-4-ethoxy-1,1,1-trifluorobut-3-en-3-one (5.0 g, 30 mmol, 1.0 eq.), cyclohexane-1,3-dione (3.36 g, 30 mmol, 1.0 eq.), ammonium acetate (4.62 g, 60 mmol, 2.0 eq.), and acetic acid (7.2 g, 120 mmol, 4.0 eq.) was heated at 100° C. for 2 h and was then allowed to cool to room temperature. Water was added and the reaction mixture was extracted with hexane. The organic fraction was dried over magnesium sulphate and the solvent was removed under reduced pressure to give 2-(trifluoromethyl)-7,8-dihydroquinolin-5(6H)-one 35 and 2-(trifluoromethyl)-5,8-dihydroquinolin-7(6H)-one (1.90 g, 29%) as a 4:1 mixture of regioisomers.

### Preparation of 2-(Trifluoromethyl)-1,6-naphthyridin-5(6H)-one 40

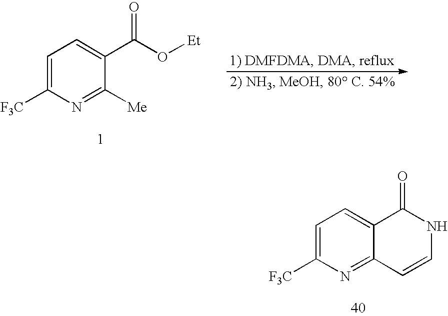

A mixture of ethyl 2-methyl-6-(trifluoromethyl)nicotinate 1 (100 g, 0.43 mol, 1.0 eq.) and N,N-dimethylformamide dimethyl acetal (53.6 g, 0.45 mol, 1.0 eq.) in N,N-dimethylformamide (300 mL) was heated at reflux overnight and then allowed to cool to room temperature. The solvent was removed under reduced pressure and the residue was treated with 18% ammonia in methanol (500 mL) at 80° C. for 2 h. The solvent was removed under reduced pressure and the residue was slurried with ethyl acetate to afford 2-(trifluoromethyl)-1,6-naphthyridin-5(6H)-one 40 (49.5 g, 54%) as a tan solid.

### Preparation of 5-Chloro-2-(trifluoromethyl)-1,6-naphthyridine 41

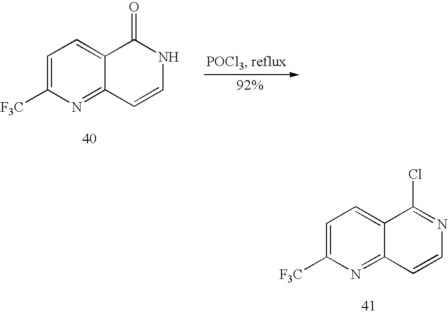

2-(Trifluoromethyl)-1,6-naphthyridin-5(6/f)-one 40 (15.0 g, 70.0 mmol) was heated at reflux with phosphorus oxychloride (50 mL, 0.54 mol) for 30 min and the reaction mixture was then slowly poured into water whilst the temperature of the reaction mixture was maintained between 20-30° C. The aqueous phase was extracted with dichloromethane, the organic phase was washed with water (2×), saturated aqueous sodium hydrogen carbonate, and saturated sodium chloride, dried over magnesium sulphate, and the solvent was removed under reduced pressure to afford 5-chloro-2-(trifluoromethyl)-1,6-naphthyridine 41 (15.0 g, 92%) as a solid, m.p. 90-90° C.

### Preparation of 8-Bromo-2-(trifluoromethyl)-1,6-naphthyridin-5(6H)-one 44

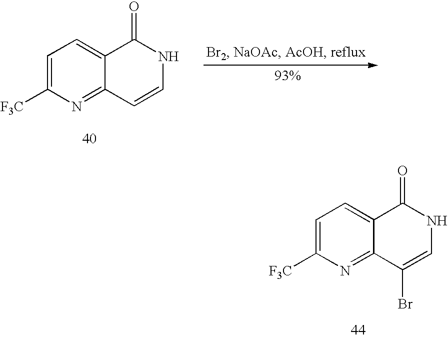

A solution of bromine (14.94 g, 93.4 mmol, 1.0 eq.) in acetic acid (10 mL) was added dropwise to a stirred solution of 2-(trifluoromethyl)-1,6-naphthyridin-5(6H)-one 40 (20.0 g, 93.4 mmol, 1.0 eq.) in acetic acid (200 mL) and upon complete addition, the reaction mixture was allowed to stir at room temperature for 30 min before being heated at reflux for 2 h. Once the reaction mixture had cooled to room temperature, water (200 mL) was added and the resultant precipitate was filtered off and air-dried. The product was then taken up in ethyl acetate, the organic phase was washed with water (2×), saturated aqueous sodium hydrogen carbonate, saturated aqueous sodium chloride, dried over magnesium sulphate, and the solvent was removed under reduced pressure to afford 8-bromo-2-(trifluoromethyl)-1,6-naphthyridin-5(6H)-one 44 (25.4 g, 93%) as a pale yellow solid, m.p. 223° C. (dec.).

### Preparation of 8-Bromo-5-chloro-2-(trifluoromethyl)-1,6-naphthyridine 58

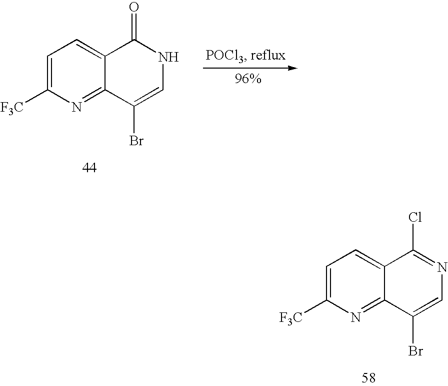

8-Bromo-2-(trifluoromethyl)-1,6-naphthyridin-5(6#)-one 44 (10.0 g, 70.0 mmol) was heated at reflux with phosphorus oxychloride (100 mL, 1.08 mol) for 90 min and the reaction mixture was then slowly poured into water (400 mL) whilst the temperature of the reaction mixture was maintained between 20-30° C. The aqueous phase was extracted with dichloromethane, the organic phase was washed with water (2×), saturated aqueous sodium hydrogen carbonate, and saturated sodium chloride, dried over magnesium sulphate, and the solvent was removed under reduced pressure to afford 8-bromo-5-chloro-2-(trifluoromethyl)-1,6-naphthyridine 58 (10.2 g, 96%) as a tan solid, m.p. 59-60° C.

### Preparation of [2-Methyl-6-(trifluoromethyl)pyridin-3-yl](phenyl)methanone

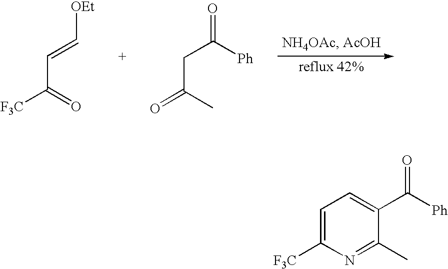

A mixture of (3E)-4-ethoxy-1,1,1-trifluorobut-3-en-3-one (5.0 g, 30 mmol, 1.0 eq.), 1-phenylbutane-1,3-dione (4.87 g, 30 mmol, 1.0 eq.), ammonium acetate (4.62 g, 60 mmol, 2.0 eq.), and acetic acid (18 g, 0.3 mol, 10 eq.) was heated at reflux overnight and then allowed to cool to room temperature. Water was added and the reaction mixture was extracted with dichloromethane. The organic fraction was dried over magnesium sulphate and the solvent was removed under reduced pressure to give [2-methyl-6-(trifluoromethyl)pyridin-3-yl](phenyl)methanone 33 (3.3 g, 42%).

### Preparation of Diethyl 6-Methylpyridine-2,5-dicarboxylate

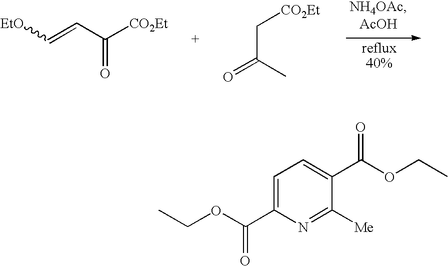

A mixture of 4-ethoxy-2-oxobut-3-enoate (5.0 g, 29.0 mmol, 1.0 eq.) (note: for the synthesis of 4-ethoxy-2-oxobut-3-enoate see below), ammonium acetate (11.2 g, 0.145 mol), and ethyl 3-oxobutanoate (3.78 g, 29.0 mmol, 1.0 eq.) in acetic acid (50 mL) was heated at reflux for 1 h and then allowed to cool to room temperature. The reaction mixture was then poured into water (200 mL) and extracted with dichloromethane (100 mL). The organic phase was washed with water (2×100 mL), saturated aqueous sodium hydrogen carbonate (2×), and saturated aqueous sodium chloride, dried over magnesium sulphate, and the solvent was removed under reduced pressure to afford diethyl 6-methylpyridine-2,5-dicarboxylate (2.80 g, 40%) as a dark oil.

### Preparation of 4-Ethoxy-2-oxobut-3-enoate (G. Dujardin, S. Rossignol, E. Brown Synthesis 1998, 763)

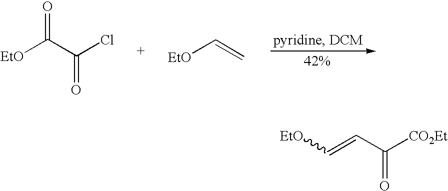

Ethyl vinyl ether (5.0 g, 69.0 mmol, 1.0 eq.) was added dropwise to a solution of ethyl chloro(oxo)acetate (9.42 g, 69.0 mmol, 1.0 eq.) and pyridine (5.47 g, 69.0 mmol, 1.0 eq.) in dichloromethane (100 mL) at 0° C. under an atmosphere of argon. Upon complete addition, the reaction mixture was stirred at 0° C. for 30 min and was then allowed to warm to room temperature. Water (100 mL) was added and the two phases were separated. The organic phase was washed with saturated aqueous sodium hydrogen carbonate (3×100 mL) and saturated aqueous sodium chloride, dried over magnesium sulphate, and the solvent was removed under reduced pressure to give ethyl 4-ethoxy-2-oxobut-3-enoate (5.0 g, 42%) as an orange oil and a 2:1 mixture of geometrical isomers.

### Preparation of 1-[2-Methyl-6-(trifluoromethyl)pyridin-3-yl]ethanone

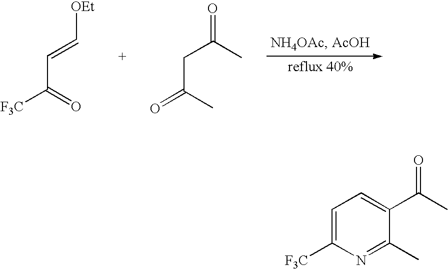

A mixture of (3E)-4-ethoxy-1,1,1-trifluorobut-3-en-3-one-(5.0 g, 30 mmol, 1.0 eq.), penta-2,4-dione (2.98 g, 30 mmol, 1.0 eq.), ammonium acetate (4.62 g, 60 mmol, 2.0 eq.), and acetic acid (7.2 g, 120 mmol, 4.0 eq.) was heated at reflux for 2 h and then allowed to cool to room temperature. Water was added and the reaction mixture was extracted with hexane. The organic fraction was dried over magnesium sulphate and the solvent was removed under reduced pressure to afford 1-[2-methyl-6-(trifluoromethyl)pyridin-3-yl]ethanone (2.7 g, 40%).

### Preparation of 2-Amino-6-(trifluoromethyl)nicotinamide 24

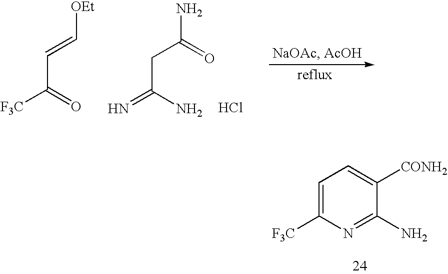

A mixture of 3-amino-3-iminopropanamide hydrochloride (20.0 g, 0.145 mol), (3E)-4-ethoxy-1,1,1-trifluorobut-3-en-2-one (24.4 g, 0.145 mol), and sodium acetate (39.5 g, 0.290 mol) was heated at reflux overnight. After being allowed to cool to room temperature, the mixture was partitioned between water (100 mL) and EtOAc (200 mL). The organic fraction was separated, washed with water (3×100 mL), saturated sodium bicarbonate solution, and brine, dried (MgSO₄), and concentrated under reduced pressure. The residue was then allowed to form a slurry in isopropyl alcohol (50 mL), and the resultant precipitate was collected by filtration and dried to yield 2-amino-6-(trifluoromethyl)nicotinamide 24 as a pale yellow solid, mp 227-229° C.

### Preparation of 7-(Trifluoromethyl)pyrido[2,3-d]pyrimidin-4-ol 65

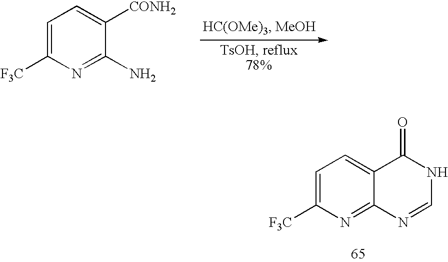

A mixture of 2-amino-6-(trifluoromethyl)nicotinamide 2008952 (5.0 g, 24.0 mmol), trimethyl orthoformate (50 mL, 456 mmol), and jc-toluenesulfonic acid (5 mg, catalytic) in methanol (50 mL) was heated at reflux overnight. After being allowed to cool to room temperature, die solvent was removed under vacuum. The residue was then allowed to form a slurry in ethyl acetate and the resultant precipitate was collected by filtration and dried to give 7-(trifluoromethyl)pyrido[2,3-d]pyrimidin-4-ol 65 (4.10 g, 78%) as a pale yellow solid, mp 268-269° C.

1.-22. (canceled)

23. A compound, or derivative thereof, of formula I 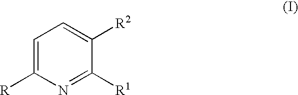
wherein R is a fluorinated C1-6 alkyl, COR³, CO₂R³, (CH₂)nCO₂R³ or (CH₂)nOR³ group optionally substituted by one or more of hydrogen, C1-6 alkyl or C1-6 haloalkyl, and wherein
n is 0 to 6;
R¹ and R² together are a group of formula (II) 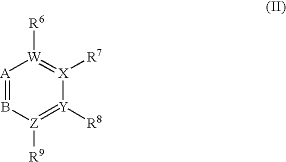
wherein A and B, which form a bicyclic fused ring system with the ring of formula I, are C;
one or two of W, X, Y or Z is N; R⁶, R⁷, R⁸ or R⁹ are independently selected from hydrogen, halogen, ═O, ═S, B(OR¹²)₂, C1-12 alkyl, C1-12 haloalkyl, cyclohydrocarbyl, heterocyclyl, OR¹², SR¹², NR¹²₂, NO₂, CN, NR¹²COR¹², NRCONR¹²₂, NRCOR¹², NR¹²CO₂R¹², S(O)₂R¹², SONR¹²₂, S(O)R¹², SO₂NR¹²₂, NR¹²S(O)₂R¹², COR¹², CO₂R¹², (CH₂)nOR¹², (CH₂)nCO₂R¹² or CONR¹²₂ optionally substituted by one or more of halogen, C1-6 alkyl, CO₂R¹³, (CH₂)nOR¹³, (CH₂)nCO₂R¹³, NR¹³R¹³ or heterocyclyl optionally substituted by NH₂;
wherein each saturated carbon in R⁶, R⁷, R⁸ or R⁹ is further optionally and independently substituted by ═O, ═S, ═NR¹⁴, NNR¹⁴₂ or —NOR¹⁴; or wherein any two of R⁶, R⁷, R⁸ or R⁹ form a partially saturated, unsaturated or fully saturated optionally substituted five or six membered ring containing zero to three heteroatoms;
wherein R¹² which may be the same or different is hydrogen, halogen, CN, OR¹⁵, CO₂R¹⁵, NR¹⁵R¹⁵, C1-6 alkyl or heterocyclyl;
wherein R¹³ which may be the same or different is hydrogen, halogen, CN, OR¹⁶, NR¹⁶R¹⁶, optionally substituted C1-12 alkyl;
R¹⁴, R¹⁵ and R¹⁶ are hydrogen or OH; and wherein n is 1 to 6.
or a pharmaceutically acceptable salt, and other pharmaceutically acceptable derivatives thereof.

24. The compound as claimed in claim 23 wherein R is fluorinated methyl or ethyl or is CO₂R³ wherein R³ is hydrogen or C1-6 alkyl.

25. The compound as claimed in claim 24 wherein R is CF₃ or CH₂F.

26. The compound as claimed in claim 23, 24, or 25 wherein R⁶, R⁷, R⁸ or R⁹ is independently selected from ═O, CN, halogen, COR¹², CO₂R¹², NR¹²R¹², B(OR¹²)₂, (CH₂)nCO₂R¹², C1-6 alkyl, heterocyclyl optionally substituted by one or more of NR¹³R¹³ or heterocyclyl; wherein each saturated carbon in R⁶, R⁷, R⁸ or R⁹ is further optionally and independently substituted by ═O, ═S, ═NR¹⁴ or ═NOR¹⁴; wherein R¹² is hydrogen, halogen, NR¹⁵R¹⁵, C1-6 alkyl, or heterocyclyl; wherein R¹³, R¹⁴ and R¹⁵ are hydrogen and wherein n is 1 or 2.

27. The compound as claimed in claim 26 wherein the R⁶, R⁷, R⁸ or R⁹ is optionally substituted piperidine or piperazine.

28. A process for the manufacture of a compound of formula I 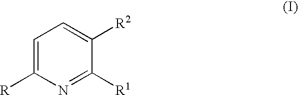
wherein R and R¹ are the same or different and R is a fluorinated C1-6 alkyl, COR³, CO₂R³, (CH₂)nCO₂R³ or (CH₂)nOR³ group optionally substituted by one or more of hydrogen, C1-6 alkyl or C1-6 haloalkyl;
R¹ is NR³R³ or hydrocarbyl optionally substituted by one or more of halogen, CO₂R⁴, OR⁴, (CH₂)nOR⁴, (CH₂)nCO₂R³, NR⁴R⁴ or optionally substituted C1-6 alkyl;
R² is halogen, C1-6 alkyl, NO₂, CN, S(O)₂R³, COR³, CO₂R³, (CH₂)nOR³, (CH₂)nCO₂R³ or CONR³₂ optionally substituted by one or more of halogen, OR⁴, CN, C1-6 alkyl, CO₂R⁴, (CH₂)nOR⁴, (CH₂)nCO₂R³ or NR⁴R⁴; wherein each saturated carbon in R² is further optionally and independently substituted by ═O, ═S, ═NR⁵, NNR⁵₂ or ═NOR⁵;
or R¹ and R² together form a partially saturated, unsaturated or fully saturated five or six membered ring containing zero to three heteroatoms which is further optionally fused to another partially saturated, unsaturated or fully saturated five or six membered ring to form a ring system containing zero to three heteroatoms, and each substitutable carbon atom in the optionally fused ring(s) or ring system(s) is optionally and independently substituted by one or more of halogen, ═O, ═S, C1-12 alkyl, C1-12 haloalkyl, cyclohydrocarbyl, heterocyclyl, OR³, SR³, NR³₂, NO₂, CN, NR³COR³, NRCONR³₂, NRCOR³, NR³CO₂R³, S(O)₂R³, SONR³₂, S(O)R³, SO₂NR³₂, NR³S(O)₂R³, COR³, CO₂R³, (CH₂)nOR³, (CH₂)nCO₂R³ or CONR³₂optionally substituted by one or more of halogen, optionally substituted C1-6 alkyl, CO₂R⁴, (CH₂)nOR⁴, (CH₂)nCO₂R⁴ or NR⁴R⁴;
R³ which may be the same or different and is hydrogen, halogen (Cl, F, I or Br), CN, OR⁵, CO₂R⁵, (CH₂)nNR⁵R⁵, NR⁵R⁵, (CH₂)nOH, C1-6 alkyl, heterocyclyl or aryl;
R⁴ which may be the same or different is hydrogen, halogen, CN, OR⁵, (CH₂)nNR⁵R⁵, NR⁵R⁵, optionally substituted C1-12 alkyl, heterocyclyl or aryl;
R⁵ which may be the same or different is hydrogen, halogen, C1-6 alkyl or C1-6 haloalkyl;
wherein n is 0 to 6;
the process comprising reacting a compound of formula IV with a compound of formula V or VI optionally in the presence of an ammonia source 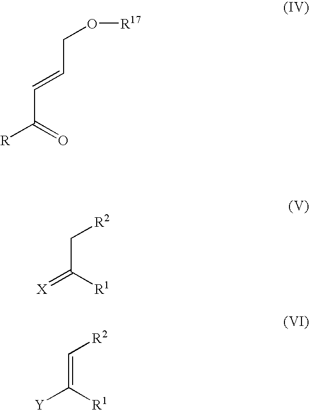
wherein R and R¹ are the same or different and R is a fluorinated C1-6 alkyl, COR³, CO₂R³, (CH₂)nCO₂R³ or (CH₂)nOR³ group optionally substituted by one or more of hydrogen, C1-6 alkyl or C1-6 haloalkyl;
R¹ is NR³R³ or hydrocarbyl optionally substituted by one or more of halogen (F, Cl, Br, I), CO₂R⁴, OR⁴, (CH₂)nOR⁴, (CH₂)nCO₂R³, NR⁴R⁴ or optionally substituted C1-6 alkyl;
R² is halogen, C1-6 alkyl, NO₂, CN, S(O)₂R³, COR³, CO₂R³, (CH₂)nOR³, (CH₂)nCO₂R³ or CONR³₂ optionally substituted by one or more of halogen (F, Cl, Br, I), OR⁴, CN, C1-6 alkyl, CO₂R⁴, (CH₂)nOR⁴, (CH₂)nCO₂R³ or NR⁴R⁴; wherein each saturated carbon in R² is further optionally and independently substituted by ═O, ═S, ═NR⁵, NNR⁵₂ or ═NOR⁵;
wherein R¹ and R² together optionally form a partially saturated, unsaturated or fully saturated five or six membered ring containing zero to three heteroatoms which is further optionally fused to another partially saturated, unsaturated or fully saturated five or six membered ring to form a ring system containing zero to three heteroatoms, and each substitutable carbon atom in the optionally fused ring(s) or ring system(s) is optionally and independently substituted by one or more of halogen (Cl, I, F or Br), ═O, ═S, C1-12 alkyl (e.g C1-6 alkyl), C1-12 haloalkyl, cyclohydrocarbyl, heterocyclyl, OR³, SR³, NR³₂, NO₂, CN, NR³COR³, NRCONR³₂, NRCOR³, NR³CO₂R³, S(O)₂R³, SONR³₂, S(O)R³, SO₂NR³₂, NR³S(O)₂R³, COR³, CO₂R³, (CH₂)nOR³, (CH₂)nCO₂R³ or CONR³₂ optionally substituted by one or more of halogen (F, Cl, Br, I), optionally substituted C1-6 alkyl, CO₂R⁴, (CH₂)nOR⁴, (CH₂)nCO₂R⁴ or NR⁴R⁴;
R³ which may be the same or different and is hydrogen, halogen (Cl, F, I or Br), CN, OR⁵, CO₂R⁵, (CH₂)nNR⁵R⁵, NR⁵R⁵, (CH₂)nOH, C1-6 alkyl (e.g methyl or ethyl), heterocyclyl or aryl;
R⁴ which may be the same or different is hydrogen, halogen, CN, OR⁵, (CH₂)nNR⁵R⁵, NR⁵R⁵, optionally substituted C1-12 alkyl (e.g C1-6), heterocyclyl or aryl;
R⁵ which may be the same or different is hydrogen, halogen (Cl, F, I or Br), C1-6 alkyl or C1-6 haloalkyl;
wherein n is 0 to 6;
R¹⁷ is hydrocarbyl;
X is O, NR¹⁸ or NR¹⁸₂ in protonated form and Y is NR¹⁸₂, wherein R¹⁸ is hydrogen;
and wherein when X is O the reaction must be carried out in the presence of a source of ammonia.

29. The process as claimed in claim 28 wherein when X is NR¹⁸₂ in protonated form, R¹ is NR¹⁹R¹⁹ or OR¹⁹ wherein R¹⁹ is hydrogen or C1-12 alkyl.

30. A pharmaceutical, nutraceutical or agrochemical agent or composition comprising one or more compound as claimed in claim 1.

31. The agent as claimed in claim 30 wherein the agent is a polymer or co-polymer.

32. The agent as claimed in claim 30 wherein the agent is a dye.

33. The agent as claimed in claim 30 wherein the agent is a peptide, peptidomimetic, amino acid or amino acid analog.
